(19) United States
(12) Patent Application Publication
(10) Pub. No.: US 2024/0150712 A1
(43) Pub. Date: May 9, 2024

(54) ARTIFICIAL TARGET FOR ANTIGEN-SPECIFIC ACTIVATION AND EXPANSION OF CAR T CELLS

(71) Applicant: Miltenyi Biotec B.V. & Co KG, Bergisch Gladbach (DE)

(72) Inventors: Meike ROSKAMP, Bergisch Gladbach (DE); Cesar EVARISTO, Bergisch Gladbach (DE); Christian LANGER, Bergisch Gladbach (DE); Alexander DUNDUA, Bergisch Gladbach (DE); Anne RICHTER, Bergisch Gladbach (DE); Jonathan FAUERBACH, Bergisch Gladbach (DE)

(73) Assignee: Miltenyi Biotec B.V. & Co KG, Bergisch Gladbach (DE)

(21) Appl. No.: 18/280,706

(22) PCT Filed: Mar. 14, 2022
(86) PCT No.: PCT/EP2022/056463

§ 371 Date: Sep. 7, 2023

(30) Foreign Application Priority Data
Mar. 17, 2021 (EP) EP 21163172.6

(51) Int. Cl. C12N 5/0783; G01N 33/50; G01N 33/68

(52) U.S. Cl. CPC C12N 5/0636; G01N 33/505; G01N 33/686; C12N 2501/998

(57) ABSTRACT

The invention is directed to a method for activating CAR cells having at least one chimeric antigen binding receptor in a sample by incubating the sample with at least one substrate provided on its surface with at least one anti-CAR idiotype antibody and/or at least one antigen selected from the group consisting of CD19, CD20, CD22, BCMA, CD33, MSLN, CD123, HER2, GD2, EGFR, PSMA, MUC1, CD318, TSPAN8, CD66c, CEA, CLA, CD276, FolR1, CLEC12A, CLL-1 and CD371, and with at least one costimulatory molecule selected from the group consisting of CD28, CD2, CD6, CD26, CD53 and LFA-1 characterized in that the sample is incubated with the at least substrate in suspension thereby activating the CAR T cells to express markers selected from the group consisting of mRNA, effector molecules or cell surface activation markers.

Adoptive cell therapies such as chimeric antigen receptor (CAR) T cells, where T-cell effector functions, such as cytotoxicity, are redirected against cancer cells, are among the promising new therapy options in the rapidly evolving field of immunotherapy. Evaluation of the functional capacity of CAR T cells is performed during research & development and commercialization of the therapy, e.g. for selection of CAR binders or CAR constructs, for the quality control of CAR T cell products and for ex vivo analysis of CAR T cells persisting in treated patients One method to do this is to coculture the CAR-T cells with target cells, which express the CAR target, and measure the activation of the CAR-T cells in the presence of these cells.

### BACKGROUND

There are several major drawbacks of the use of live target cells for in vitro functional assays. They include the high variability introduced by the target cells sources used, batch to batch genetic drift, and handling variations during storage and cell culture. These represent major obstacles to bioassay standardization for functional characterization of CAR T cells. Thus, replacing live target cells with synthetic artificial reagents capable of inducing CAR T cells activation would comprise a major benefit for standardization and consistency.

Wu et al (Cellular & Molecular Immunology 2020, 17:600-612) as well as Lindner et al (Sci. Adv. 2020; 6: eaaz3223) describe in their reviews the important factors in CAR signaling when the CAR T cell interacts with a target cell: from the contact of receptors and antigens and proximal signaling to immunological synapse formation and late signaling outcomes. Several signals are required and mentioned in these papers. For this invention, the listed signals in the papers were reduced to the two signals that were needed: contact between a sufficient number of receptor and antigen as well as signaling with costimulatory signals such as CD28. Furthermore, both papers also describe that for an efficient CAR T cell activation a larger number of CARs need to be clustered.

It is also known that particles (e.g. Silica, Polystyrene, Hydrogels, PMMA to name a few) can be conjugated with antibodies and be used for various biological applications (e.g. Nano Res (2008) 1: 99 115). For this purpose, taking the example of silica particles, antibody conjugated particles (https://www.cd-bioparticles.com/products/silica-particles-list-168.html) are already commercially available. The same is also true for example for microplates and glass slides as surfaces for antibody conjugation which are available from Schott and PolyAn as functionalized microplates and glass slides for the immobilization of various biomolecules (e.g. https://www.schott.com/nexterion/english/products/functional-coatings/3d-polymer-coating.html and https://www.poly-an.de/microplates-cell-culture-consumables/microplate-surfaces). But none of these materials have ever been used to selectively target and activate CAR T cells or other CAR cells (e.g. CAR NK cells, CAR Macrophages) for that matter.

Dirar et al (PLOS ONE 15(9): e0238819) demonstrated that CAR T cells can be activated by micron-structured surfaces that are functionalized with their anti-idiotype antibody. However, even though early activation events and degranulation could be observed by microscopy, the level of activation achieved was never demonstrated to achieve biologically-relevant levels. Furthermore, they did not use anti-CD28 as a costimulatory signal. They used microcontact printing to generate a surface with micron-sized isles of anti-idiotype antibody.

### OBJECT OF THE INVENTION

Accordingly, it was an object of the invention to provide a more reliable and biologically relevant method for activating CAR T cells. This is achieved by a method for activating CAR cells having at least one chimeric antigen binding receptor in a sample by incubating the sample with at least one substrate provided on its surface with at least one anti-CAR idiotype antibody and/or at least one antigen selected from the group consisting of CD19, CD20, CD22, BCMA, CD33, MSLN, CD123, HER2, GD2, EGFR, PSMA, MUC1, CD318, TSPAN8, CD66c, CEA, CLA, CD276, FolR1, CLEC12A, CLL-1 and CD371, and with at least one costimulatory molecule selected from the group consisting of CD28, CD2, CD6, CD26, CD53 and LFA-1 characterized in that the sample is incubated with the at least substrate in suspension thereby activating the CAR T cells to express markers selected from the group consisting of mRNA, effector molecules or cell surface activation markers.

Further, the invention is directed to artificial materials (substrates) that are able to activate and preferable expand CAR cells in an antigen-dependent manner by combining the following characteristics:
(1) For binding sterically available antigen (e.g. anti-idiotype antibody or CAR antigen) covalently bound in high density on the surface of the particles or surfaces. The antigen can either be directly bound to the surface or in an indirect manner (e.g via Anti-biotin and a biotinylated antigen, or via anti-Thiamine and a Thiamination agent).
(2) Costimulatory molecule-engaging entity (e.g. Anti-CD28 antibody), either bound on the particles or surface in combination with the antigen or added in soluble form.

Another object of the invention is a substrate for activating CAR cells characterized in that at least one the substrate is provided on its surface with at least one anti-CAR idiotype antibody and/or at least one antigen selected from the group consisting of CD19, CD20, CD22, BCMA, CD33, MSLN, CD123, HER2, GD2, EGFR, PSMA, MUC1, CD318, TSPAN8, CD66c, CEA, CLA, CD276, FolR1, CLEC12A, CLL-1 and CD371, and with at least one costimulatory molecule selected from the group consisting of CD28, CD2, CD6, CD26, CD53 and LFA-1.

Applications for these materials can be the use as targets to assess, qualitatively or quantitatively, any of the biological events triggered by activation of the CAR, e.g. in quality control of manufactured CAR T cell batches, potency assays, research of relevant markers for activation, sorting of activated cells or for an antigen-specific expansion of CAR T cells for analytical or therapeutic applications.

The CAR antigens are preferable selected from the group of liquid tumor and solid tumor antigens. The method of the invention and the substrates of the invention are applicable to all sorts of CAR cells, but especially for CAR T cells, CAR NK cells and/or CAR macrophages.

### BRIEF DESCRIPTION OF THE DRAWINGS

FIG. 1 to 6 show several embodiments to provide substrates with anti-CAR idiotype antibodies, antigens and costimulatory molecules

FIG. 7 to 20 show results of the experiments according to the invention

### DETAILED DESCRIPTION

Preferable, the method of the invention is conducted by providing the substrate as particles having a mean diameter of at least 0.4 μm, preferentially with a mean diameter between 1-5 μm. The substrate may comprise silica, polystyrene, polyolefins, polysaccharides, polyesters, polyacrylates, polylactic and/or iron oxide. It is further possible to use these materials as coating of particles made from yet other materials. Most preferred, the surface is additional provided with a chemically reactive group like an carboxy or amine group.

Preferable, the particles are provided to the sample in suspension, for example suspended in cell media.

In a first embodiment, the substrate is prepared by binding the at least one anti-CAR idiotype antibody or at least one antigen and at least one costimulatory molecule to the substrate (i.e. is surface) via a primer molecule. As primer molecule, SMCC (Succinimidyl-trans-4-(N-maleimidylmethyl)cyclohexane-1-carboxylate) is preferred. Further, as primer molecule SM(PEG)n with n=2, 4, 6, 8, 12, 24 can be used.

In another embodiment, the substrate is prepared by binding the at least one anti-CAR idiotype antibody or at least one antigen and at least one costimulatory molecule to the substrate (i.e. is surface) via a biotinylated antibody. The biotinylated antibody again may be bound to the surface via a primer molecule.

Further, the at least one anti-CAR idiotype antibody and/or at least one antigen selected from the group consisting of CD19, CD20, CD22, BCMA, CD33, MSLN, CD123, HER2, GD2, EGFR, PSMA, MUC1, CD318, TSPAN8, CD66c, CEA, CLA, CD276, FolR1, CLEC12A, CLL-1 and CD371, and the at least one costimulatory molecule selected from the group consisting of CD28, CD2, CD6, CD26, CD53 and LFA-1 are provided on the surfaces of at least two different substrates, for example to two or more different particles.

In the method of the invention, the activated CAR T cells may further be detected, directly or indirect for example via secretion of messenger molecules. Detection of the activation of the CAR cells may be performed by imaging, flow cytometry or RT-PCR/RNAseq.

Alternatively, the activated CAR T cells may be detected indirectly. For example, the method may be conducted by incubating the sample with the substrates, especially the particles, in suspension thereby activating the CAR T cells to secret proteins and detecting the secreted proteins. In this variant, it is further possible to activate the CAR T cells to express markers selected from the group consisting of mRNA, effector molecules or cell surface activation markers and detect the markers. Detecting such markers or and secreted proteins is within the common knowledge of the person skilled in the art.

In contrast to biological systems like antigen-expressing target cells, artificial systems can be produced with a more defined and reproducible chemical composition, which enables a potentially more reproducible material compared to the target cells. They can also be produced at a large scale with less effort compared to cells. Another potential benefit is a better storage stability compared to target cells.

The combination of anti-idiotype antibody on an at least micron-sized surface for example with Anti-CD28 either in solution or bound together with the anti-idiotype antibody results in a strong enough activation of CAR T cells in order to be useful to replace target cells in potency assays or to be used for a CAR specific expansion of CAR T cells.

The method of the invention can be performed in the following workflows:

Variant 1: The level of IFN-γ secreted by CAR cells is determined after in vitro culture with the CAR-binding particles. The readout method for the determination of IFN-γ levels uses the Miltenyi-developed MACSPlex IFN-γ immunoassay, which is a bead-based flow cytometric cytokine detection assay. Supernatant samples collected after culture are incubated with anti-IFN-γ antibody-coated MACSPlex Capture Beads, and IFN-γ binds to the specific antibodies. A detection reagent, composed of a fluorochrome-conjugated antibodie specific for IFN-γ is added. Consequently, sandwich complexes are formed between the MACSPlex Capture Bead, IFN-γ and the detection reagent. These complexes can be analyzed by flow cytometry based on the fluorescence characteristics of both the MACSPlex Capture Bead and the detection reagent. Standards of known quantities of IFN-γ are used for the quantification of the cytokine in the supernatant.

Variant 2: The frequency of IFN-γ-expressing CAR cells is determined after in vitro culture with the CAR-binding particles in the presence of a protein transport inhibitor, such as Brefeldin A. This results in accumulation of IFN-γ in the cytoplasm after CAR cell activation. After culture, cells are fixed, e.g. with paraformaldehyde and the cell membrane is permeabilized, e.g. with saponin, in order to allow the detection reagent to penetrate the cytoplasm. Fluorochrome-conjugated antibodies specific for IFN-γ are added, resulting in the fluorescent labeling of the cells which were activated to express IFN-γ during the culture. Additional fluorescent-conjugated antibodies can be added to detect expression of additional cytokines, or the expression of surface activation induced proteins, such as CD137. The cells can be analyzed by flow cytometry, based on the optical and fluorescence characteristics of the labeled cells.

### EXAMPLES

Artificial targets were prepared by covalently binding anti-Biotin antibody to the surface of silica beads, followed by an immobilization of a mixture of biotinylated anti-idiotype antibody (either anti-CD19 CAR, named C01, or anti-CD20 CAR, named 37B4) and biotinylated anti-CD28 on the surface of the anti-biotin coated silicaparticles.

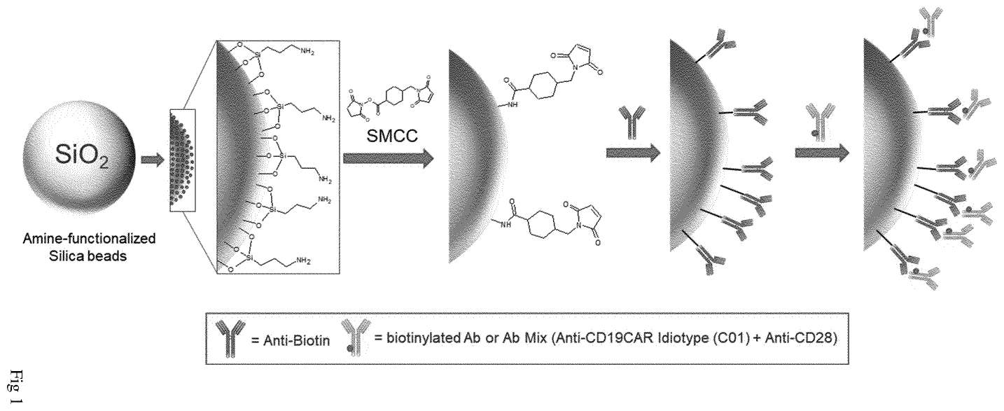

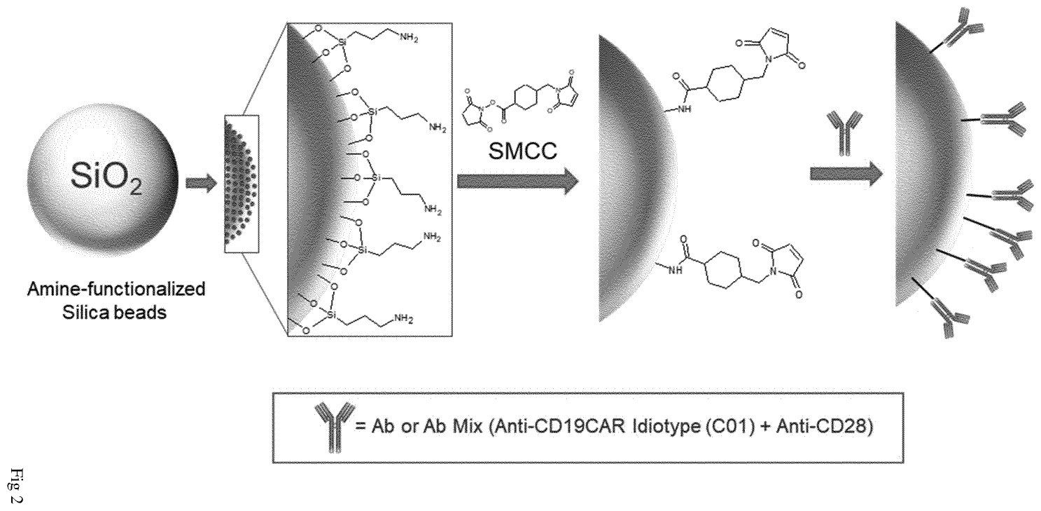

A scheme of the preparation is shown in FIG. 1 or 2.

For the covalent attachment of anti-Biotin antibody to the surface of silica beads, amine functionalized silica beads were first activated with SMCC by adding SMCC in DMSO to the amine functionalized silicabeads in water. The mixture was incubated for 1.5 hours at r.t. before the beads were purified by ultrafiltration in Vivaspin filtration units (MWCO 100 kDa, 8 wash cycles, 6 cycles with water followed by 2 cycles with PBS). The concentration of the now maleimide-functionalized silicabeads after purification was ca. 10 g/L in PBS. For the antibody conjugation to the maleimide functionalized silicabeads, anti-Biotin antibody was reduced by incubation of a 4.5 g/L antibody solution with 10 mM DTT in 50 mM MES buffer pH6 for 1 hour followed by purification and buffer exchange into PBS using PD10 columns. Than the reduced anti-Biotin antibody was added to the maleimide-functionalized silicabeads (50 μg reduced antibody per mg silicabead, ca. 11 equivalents antibody per maleimide group) and the mixture was incubated at 4° C. for 2 days. Afterwards the particles were purified by centrifugation (2 centrifugation steps at 1000 g for 5 min, PBS was used as washing buffer) and resuspended to a final particle concentration of ca. 12 g/L in PBS. The obtained ratio of antibody per mg silicabead was dependent on the used silicabead size (for example 3.9 μm silicabeads=0.81 μg anti-Biotin per mg silica; 2 μm silicabeads=1.2 μg anti-Biotin per mg silica).

For the immobilization of the mixture of biotinylated anti-idiotype and anti-CD28 antibody, 50 μL of a mixture of both biotinylated antibodies and the anti-Biotin functionalized silicabeads in TexMACS medium was incubated at r.t. for 15 min. The molar ratio of anti-idiotype to anti-CD28 antibody was 3.3:1. The concentration of antibodies in the reaction mixture was 4 μg/ml anti-idiotype, 1.2 μg/ml Anti-CD28 and 1.2 μg/ml Anti-biotin antibody (conjugated on the silicabeads). This material was used without further purification.

The same functionalization chemistry that was performed on the silica beads was also performed on amine-functionalized microplates, which were purchased from PolyAn.

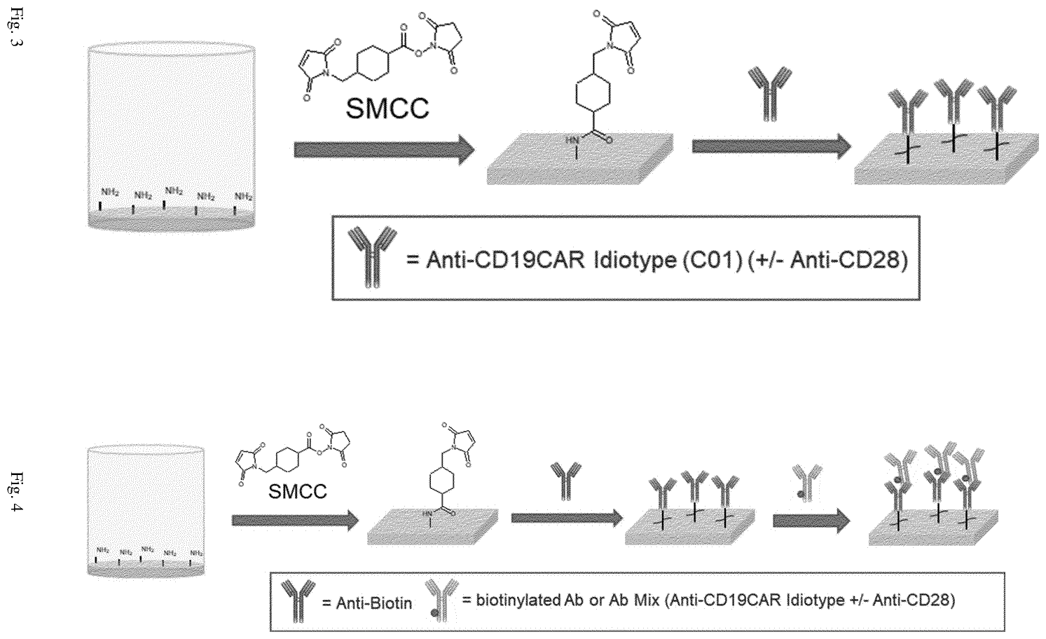

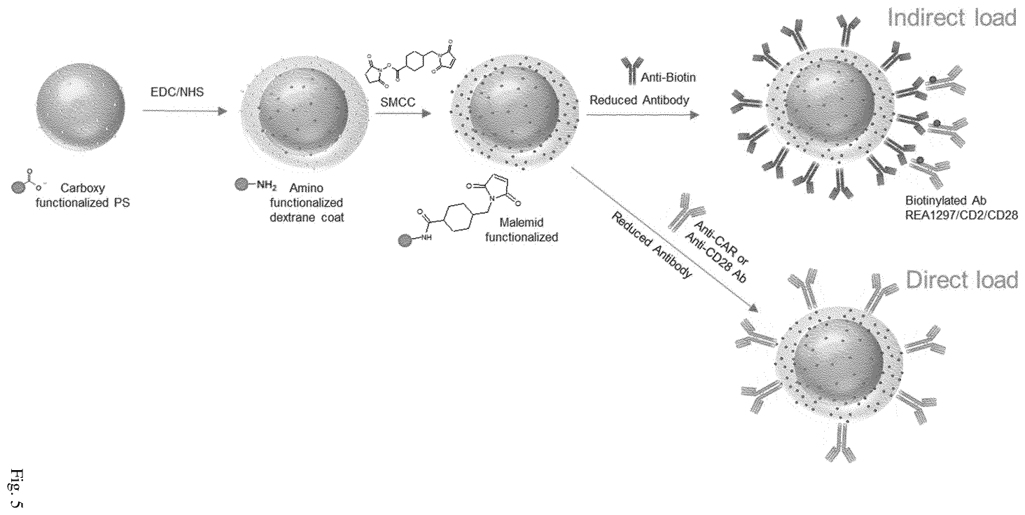

A scheme of the preparation is shown in FIGS. 3 and 4

Using a different substrate, carboxy functionalized polystyrene beads were activated with DCH/NHS and then coated with Amino-Dextran molecules and finally maleimide functionalized by modification with SMCC, after which reduced antibodies were covalently coupled to the beads

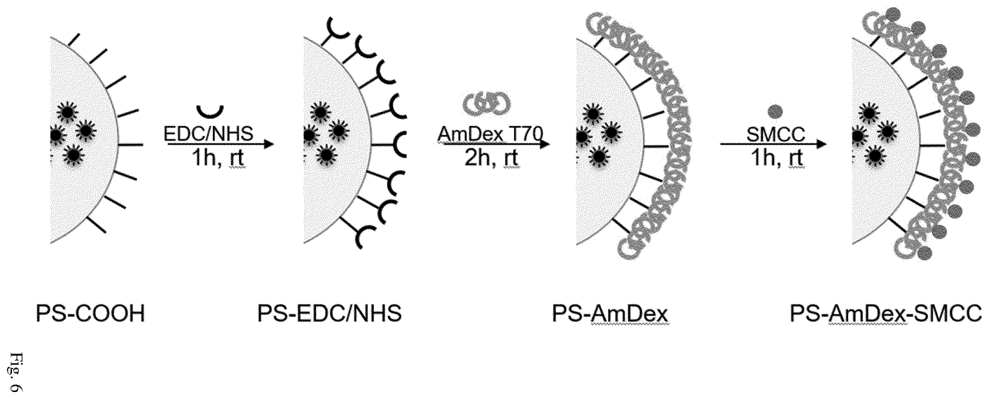

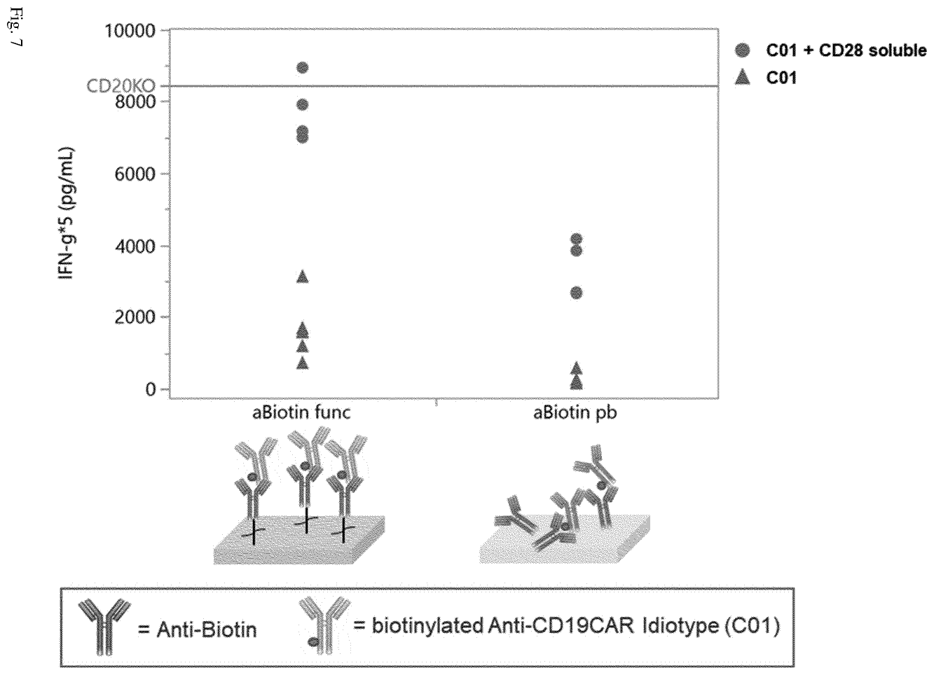

A scheme of the preparation is shown in FIGS. 5 and 6.

The different functionalized materials were tested in a functional bioassay. CD2019 CAR T cells (T cells expressing a tandem CAR with antigen binding domains for CD20 and CD19) were cultured in the presence of the different materials (artificial target materials or Jeko-1 control target cell line) overnight at 37° C. Unless stated otherwise, the antibody concentrations during cell culture with the different materials were 1 μg/ml anti-idiotype and 0.3 μg/ml Anti-CD28 antibody. Secretion of effector molecules to the supernatant was then measured using MACSPlex. In the figures IFNγ is shown as an indicator of CAR T cell activation.

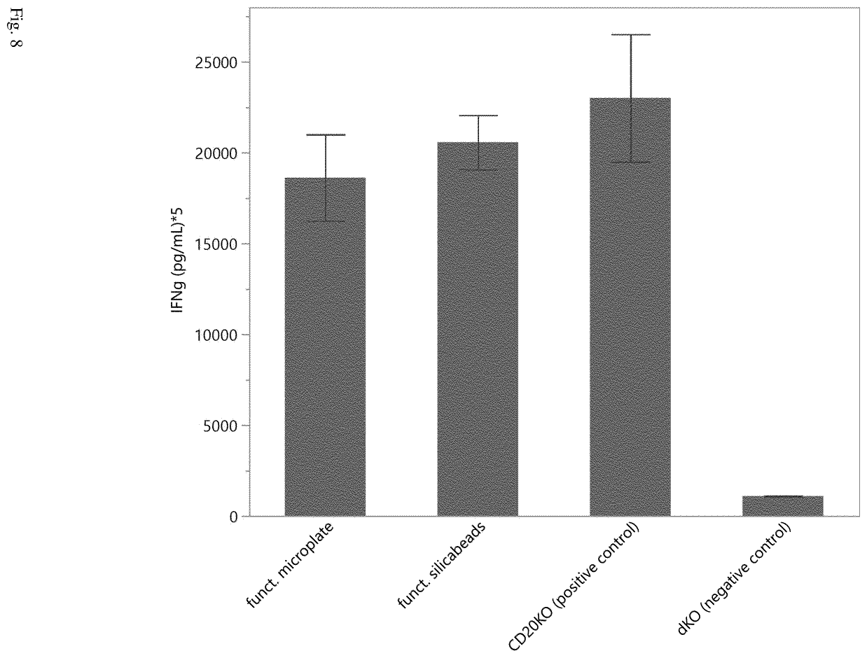

Comparing microplates with adsorbed antibody (which generally have a lower degree of functionalization compared to a highly covalently modified plate due to possible desorption and also have a risk of unfavorable orientation of the antibody for the next binding step) versus microplates with covalently attached antibody (which were conjugated with the highest possible amount of antibody) it could be shown that an antigen-specific activation could be achieved with both but to a higher extent with the covalently functionalized microplate. It could also be shown that the addition of a costimulatory molecule, in this case anti-CD28, greatly enhances the activation of the CAR T cells compared to the samples that only contain the anti-idiotype antibody. FIG. 7 shows the results.

This comparison also illustrates that a high density is favorable for a good activation of the CAR T cells.

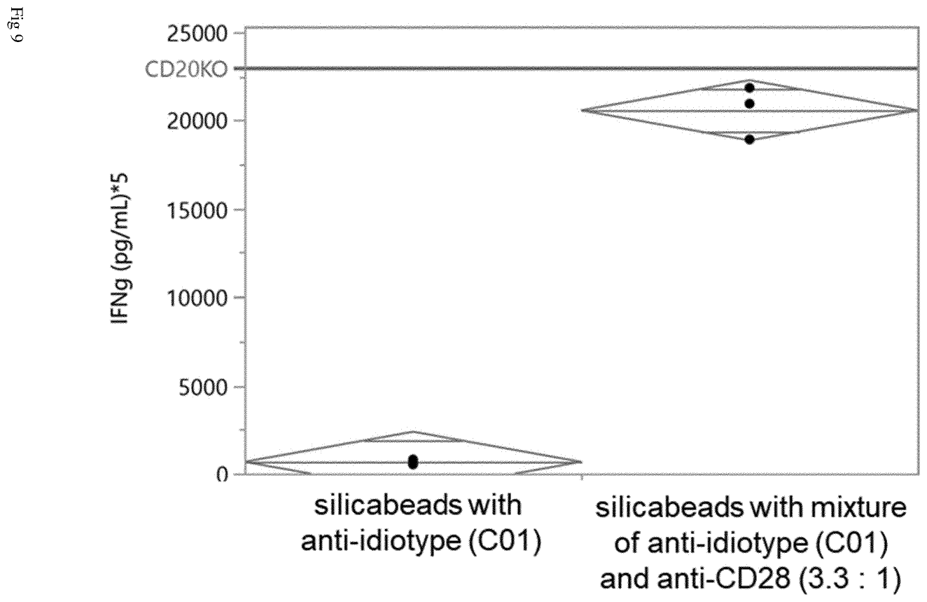

The comparison of covalently modified microplates with functionalized particles showed that micron-sized silica or polystyrene particles functionalized with a mixture of anti-idiotype antibody and anti-CD28 and an average size around 4 μm work equally well to activate CAR T cells as microplates with an identical surface coating, resulting in similar levels of IFNγ. After the co-culture with CAR T cells, both materials result in a similar level of IFNγ as the control target cells (CD20 knockout Jeko-1 cells, named CD20KO; CD19 knockout Jeko-1 cells, named CD19KO; CD20 and CD19 double knockout Jeko-1 cells, named dKO), demonstrating their usability as artificial targets in the assay and potentially also for CAR T cell expansion. FIG. 8 shows the results.

The addition of Anti-CD28 in combination with the anti-idiotype antibody enhanced the activation significantly compared to the use of the anti-idiotype antibody alone, demonstrating the importance of having a costimulatory signal in order to obtain activation in the range of target cells with the artificial targets.

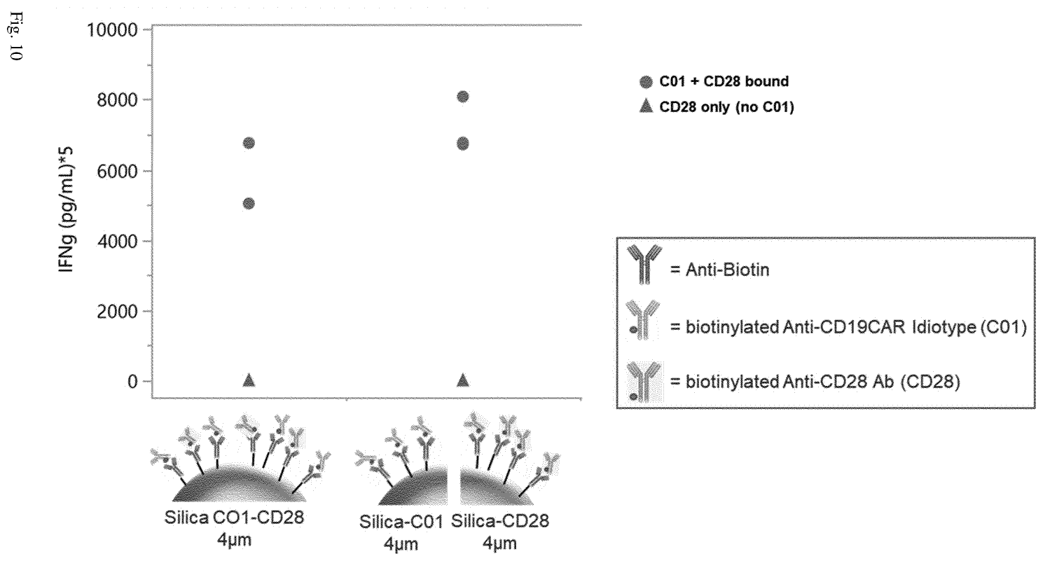

FIG. 9 shows the comparison of activation with anti-idiotype alone versus the combination of anti-idiotype with anti-CD28 at a ratio of 3.3:1.

The mixture of anti-idiotype antibody and anti-CD28 can be prepared by mixing both antibodies during immobilization of the biotinylated antibodies on the anti-Biotin coated silica beads as described above, which results in beads which have both antibodies on their surface at the desired ratio. Alternatively, silica beads can be prepared with the individual antibodies, and then the silica beads with one antibody type each on their surface are mixed in the desired ratio of anti-idiotype antibody to anti-CD28. Both materials result in a similar level of CAR T cell activation. The preparation of individual beads instead of beads with a mixture of antibodies can be an advantage for a quantification of the individual antibodies on the beads. Beads with only anti-CD28 on their surface (at the same anti-CD28 concentration as used in the samples with a mixture of anti-idiotype antibody and anti-CD28) are not able to activate CAR T cells, which demonstrates that the activation with the tested conditions is CAR specific. FIG. 10 shows the results.

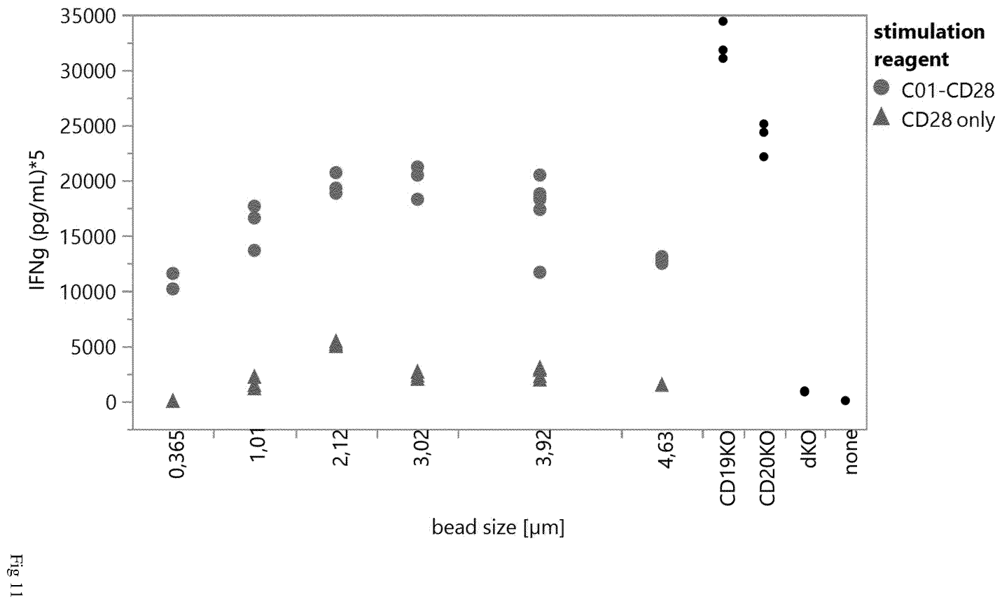

A range of silica bead sizes can be used as artificial targets. Particles with diameters from ca. 0.4 μm up to ca. 4.6 μm were tested. The ideal bead size was shown to be in the range between 2 and 4 μm, but all tested particle sizes were able to activate the CAR T cells. It was also shown that for all particle sizes at comparable anti-CD28 concentration (in absence of the idiotype antibody) very low levels of activation are observed, thereby demonstrating that the observed activation of the CAR T cells is indeed antigen specific and is not achieved with anti-CD28 alone. FIG. 11 shows the results.

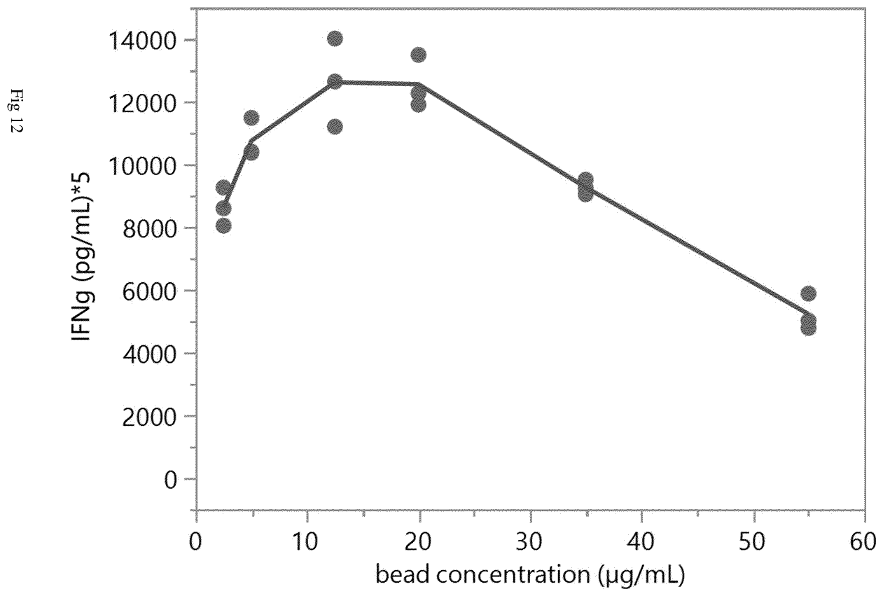

The activation of CAR T cells with the artificial targets is concentration dependent. The ideal concentration will vary for different CAR T cells, for the CAR T cells tested in the shown examples, the ideal concentration of anti-idiotype antibody (immobilized on silicabeads together with anti-CD28) during activation was in the range of 5-25 μg/mL. FIG. 12 shows the results.

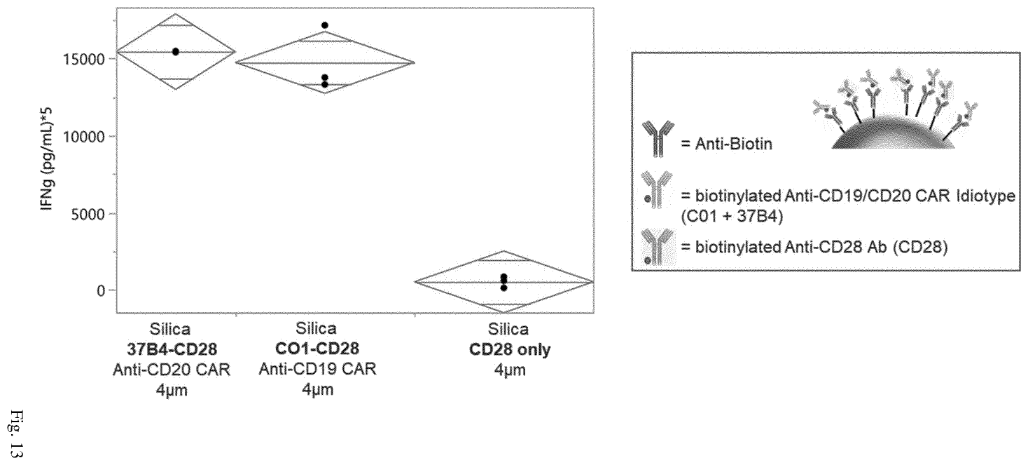

An activation with the artificial target could not only be achieved by targeting the CD19 CAR of the CD2019 CAR T cells, but also by targeting the CD20 CAR with anti-CD20 CAR idiotype antibody (named 37B4), thereby demonstrating that the same concept can be used for other CAR specificities as well. FIG. 13 shows the results.

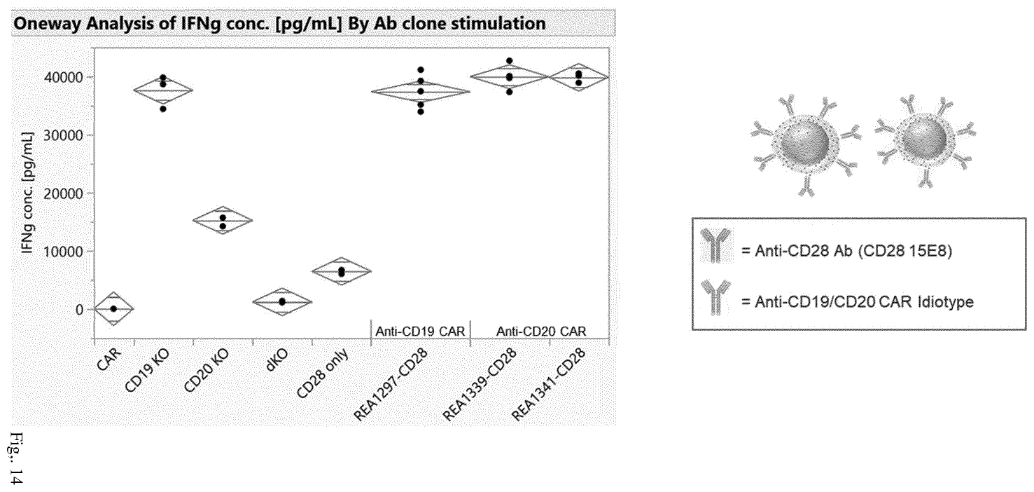

Polystyrene-based artificial targets, described above and depicted in FIG. 5 consisting of a mixture of either anti-CD19 CAR idiotype antibody (clone REA1297) or anti-CD20 CAR idiotype antibody (REA1339 or REA1341) beads and anti-CD28 antibody (clone 15E8) beads were used to activate CAR T cells specific to CD20 and to CD19. As positive controls, CAR T cells were co-cultured with Jeko-1 cells expressing CD20 (“CD19KO”) or CD19 (“CD20KO”). As negative controls, CAR T cells were cultured either alone (“CAR”), with Jeko-1 cells that don't express CD20 or CD19 (“dKO”), or CAR T cells cultured with anti-CD28 antibody (clone 15E8) beads (“CD28 only”). Activation was assessed by measuring the concentration of INFγ secreted to the supernatant. As FIG. 14 shows, artificial targets activation resulted in CAR T cell activation and IFNγ secretion that reached similar concentrations as activation by CD20-expressing JeKo-1 cells (“CD19KO”). These results demonstrate activation using artificial targets to be comparable to activation by cellular targets.

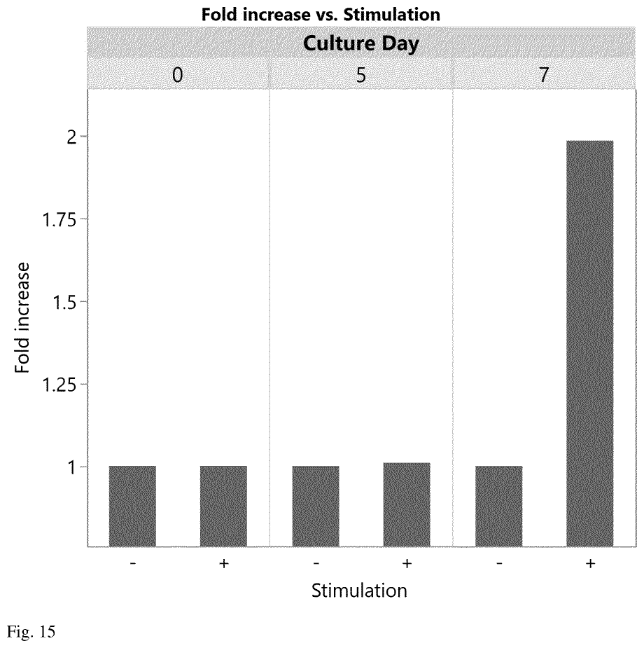

CAR T cells were co-cultured in TexMACS medium supplemented with human AB serum, IL-7, IL15 and Pen/Strep, with or without polystyrene-based artificial targets and enumerated after 5 and 7 days to assess expansion. As shown in FIG. 15, after 7 days the number of CAR T cells cultured with artificial targets was 2-fold higher than the number of CAR T cells cultured without artificial targets, indicating artificial targets can be successfully used to expand CAR T cells.

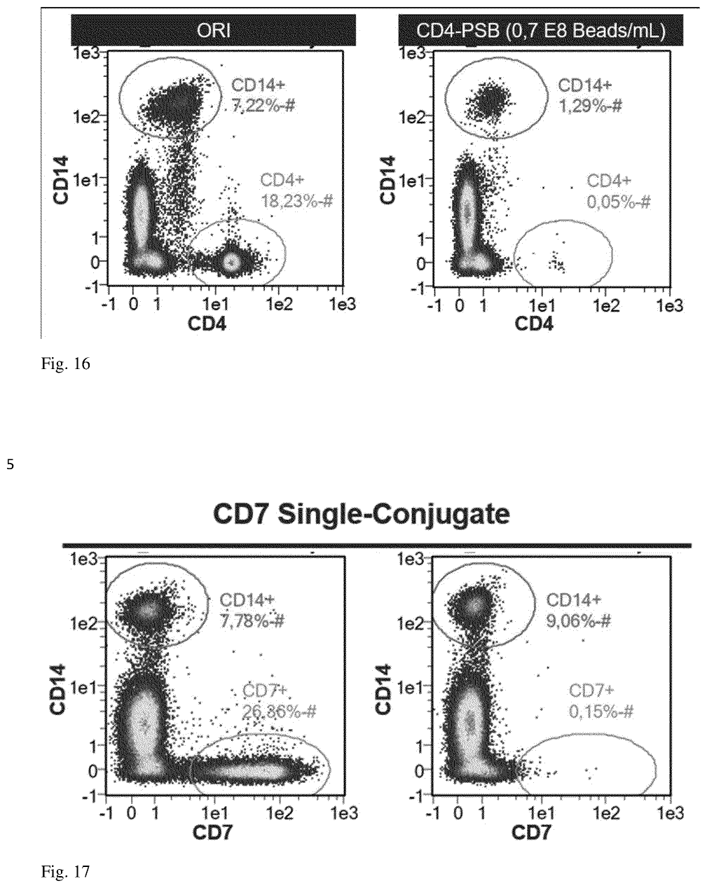

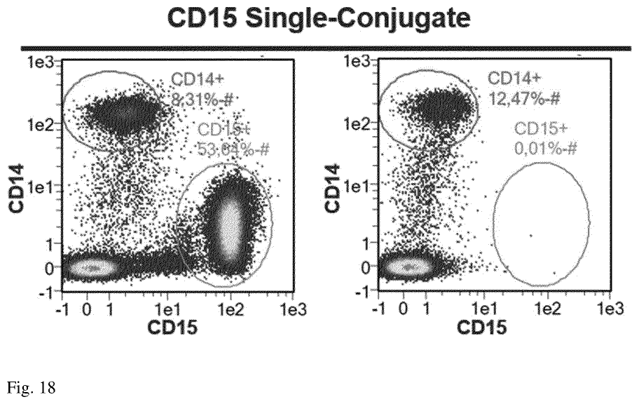

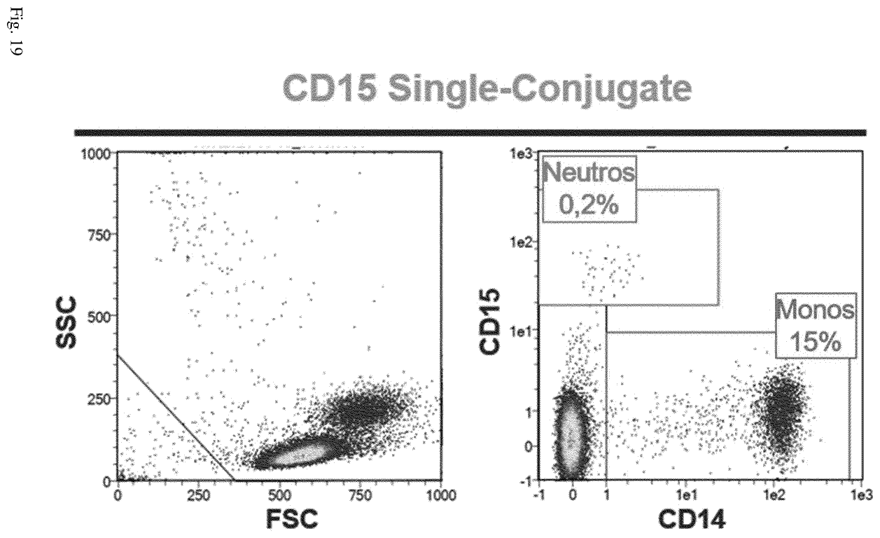

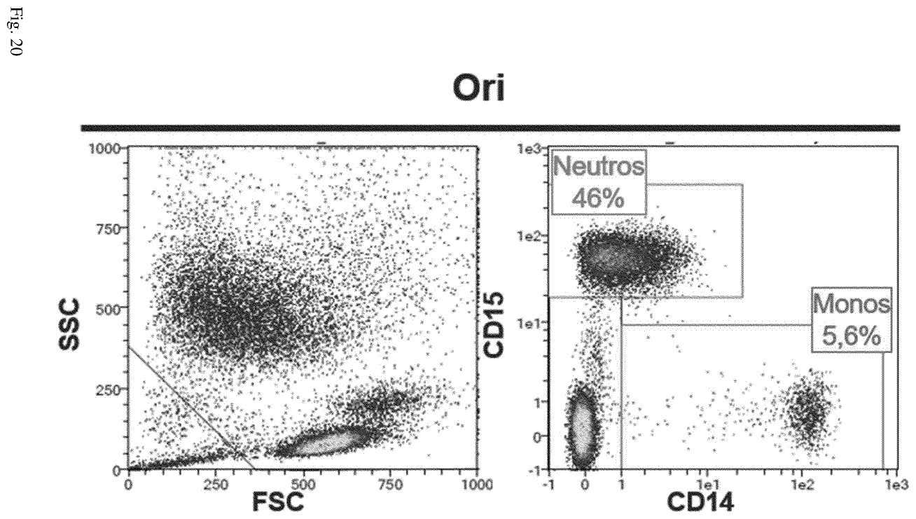

Magnetic Polystyrene beads (3 μm) were activated/coated/functionalized and coupled to a collection of dedicated Antibodies (e.g. CD4, CD15, CD7 and including anti-Biotin) incubated with human whole-blood (WB) in order to bind target cells. These cells can then be magnetically isolated and selectively depleted as seen by dot plots in FIGS. 16 to 20. This illustrates the use of polystyrene beads and magnetic polystyrene beads for activation of CAR cells but also their immunomagnetic selective labeling for manipulation, depletion and separation from, for example, Wb or culture media to name a few.

1. Method for activating CAR cells having at least one chimeric antigen binding receptor in a sample by incubating the sample with at least one substrate provided on its surface with at least one anti-CAR idiotype antibody and/or at least one antigen selected from the group consisting of CD19, CD20, CD22, BCMA, CD33, MSLN, CD123, HER2, GD2, EGFR, PSMA, MUC1, CD318, TSPAN8, CD66c, CEA, CLA, CD276, FolR1, CLEC12A, CLL-1 and CD371, and with at least one costimulatory molecule selected from the group consisting of CD28, CD2, CD6, CD26, CD53 and LFA-1 characterized in that the sample is incubated with the at least substrate in suspension thereby activating the CAR T cells to express markers selected from the group consisting of mRNA, effector molecules or cell surface activation markers.

2. The method according to claim 1 characterized in that the substrate is provided as particles in suspension wherein the particles have a mean diameter of at least 0.4 μm.

3. The method according to claim 2 characterized in that the sample is incubated with the particles in suspension thereby activating the CAR cells to secret proteins and detecting the secreted proteins.

4. The method according to claim 1 characterized in that the activation of the CAR cells is detected by imaging, flow cytometry or RT-PCR/RNAseq.

5. The method according to claim 1 characterized in that the at least one anti-CAR idiotype antibody and/or at least one antigen selected from the group consisting of CD19, CD20, CD22, BCMA, CD33, MSLN, CD123, HER2, GD2, EGFR, PSMA, MUC1, CD318, TSPAN8, CD66c, CEA, CLA, CD276, FolR1, CLEC12A, CLL-1 and CD371, and the at least one costimulatory molecule selected from the group consisting of CD28, CD2, CD6, CD26, CD53 and LFA-1 are provided on the surfaces of at least two different substrates.

6. Substrate for activating CAR cells characterized in that at least one the substrate is provided on its surface with at least one anti-CAR idiotype antibody and/or at least one antigen selected from the group consisting of CD19, CD20, CD22, BCMA, CD33, MSLN, CD123, HER2, GD2, EGFR, PSMA, MUC1, CD318, TSPAN8, CD66c, CEA, CLA, CD276, FolR1, CLEC12A, CLL-1 and CD371, and with at least one costimulatory molecule selected from the group consisting of CD28, CD2, CD6, CD26, CD53 and LFA-1.

7. Substrate according to claim 6, characterized in that the substrate is provided as particles having a mean diameter of at least 0.4 μm.

8. Substrate according to claim 6, characterized in that the particles comprise silica, polystyrene, polyolefins, polysaccharides, polyesters, polyacrylates, polylactic and/or iron oxide.

9. Substrate according to claim 6, characterized in that the at least one anti-CAR idiotype antibody and/or the at least one antigen and/or the at least one costimulatory molecule are bound to the substrate via an anti-biotin antibody, an anti-Thiamine antibody or via Streptavidin.

10. Substrate according to claim 6, characterized in that at least one anti-CAR idiotype antibody or at least one antigen and at least ne costimulatory molecule is bound to the substrate via a primer molecule.

11. Substrate according to claim 9, characterized in that the primer molecule is SMCC (Succinimidyl-trans-4-(N-maleimidylmethyl)cyclohexane-1-carboxylate)

12. Substrate according to claim 6, characterized in that the at least one anti-CAR idiotype antibody and/or at least one antigen selected from the group consisting of CD19, CD20, CD22, BCMA, CD33, MSLN, CD123, HER2, GD2, EGFR, PSMA, MUC1, CD318, TSPAN8, CD66c, CEA, CLA, CD276, FolR1, CLEC12A, CLL-1 and CD371, and the at least one costimulatory molecule selected from the group consisting of CD28, CD2, CD6, CD26, CD53 and LFA-1 are provided on the surfaces of at least two different substrates.
